(19) United States
(12) Patent Application Publication
(10) Pub. No.: US 2002/0127617 A1
(43) Pub. Date: Sep. 12, 2002

(54) Diagnostic method of hemolytic anemia

(76) Inventor: Kyung-Ja Han, Seoul (KR)

Correspondence Address: BIRCH STEWART KOLASCH & BIRCH, PO BOX 747, FALLS CHURCH, VA, 22040-0747, US

(21) Appl. No.: 10/026,542

(22) Filed: Dec. 27, 2001

(30) Foreign Application Priority Data
Dec. 29, 2000 (KR) 2001-85709

(51) Int. Cl. G01N 33/567

(52) U.S. Cl. 435/7.21

(57) ABSTRACT

This invention is related to the diagnostic method of hemolytic anemia, which allows early diagnosis of hemolytic anemia and discrimination of significant schistocytes from insignificant old schistocytes by utilizing the flow cytometric detection of damaged RBCs using anti-Hb in a hypotonic solution compared to saline solution, in the case of the presence of schistocytes in the peripheral blood, such as microangiopathic hemolytic anemia (MAHA).

This method does not require the washing or lysing steps, and get the result within 20 minutes easily. In detail, two microliter of peripheral blood isolated from a patient was stained with PE conjugated antihemoglobin (anti-Hb) antibody in 0.6% NaCl for 15 minutes at room temperature. Then, without wash, 3 ml of saline were added and analyzed by flow cytometry.

Therefore, compared with the counting schistocytes on the stained blood smears the time-consuming and hard to distinguish schistocytes from indented normal red cells (RBCs), this invention provides here a new quick and accurate diagnostic method of hemolytic anemia by flow cytometry using antihemoglobin (anti-Hb) antibody which detect only newly produced damaged RBCs and discriminate significant from insignificant old schistocytosis.

Key word: red blood cell (RBC), hemolytic anemia, red blood cell (RBC) test.

### BACKGROUND OF THE INVENTION

1. Field Of The Invention

This invention concerns novel methods for the diagnosis of Hemolytic Anemia

2. Background And Related Disclosures

This invention is related to the diagnostic method of hemolytic anemia, which allows early diagnosis of hemolytic anemia and discrimination of significant schistocytes from insignificant old schistocytes by utilizing the flow cytometric detection of damaged RBCs using anti-Hb in a hypotonic solution compared to saline solution, in the case of the presence of schistocytes in the peripheral blood, such as microangiopathic hemolytic anemia (MAHA).

The conventional method for red blood cell (RBC) test involving microscopic examination of blood smears isolated form patients, has a limit for the accurate diagnosis of hemolytic anemia, such as hard to distinguish schistocytes from indented normal red cells(RBCs), especially newly produced damaged RBCs from insignificant old schistocytosis. The presence of schistocytes in the peripheral blood is a cardinal sign of microangiopathic hemolytic anemia(MAHA), and is also important in many other diseases associated with intravascular hemolysis including disseminated intravascular coagulation. The only available method to confirm the presence of schistocytes is microscopic examination of the blood smears after Romanovsky stain, and this is the only method of the quantitative analysis of schistocytes also. However, it is labor intensive, time-consuming, and hard to distinguish schistocytes from indented normal red cells(RBCs). Sometimes, the patients, who received splenectomy revealed marked poikilocytosis including schistocytes without any significant clinical signs of hemolysis. Therefore, it is needed to develop a new method to detect and quantitate schistocytes in the peripheral blood, preferentially, newly produced schistocytes with clinical significance from the old one without clinical significance. In extravascular hemolysis, frequently poikilocytosis including spherocytosis and elliptocytosis is associated, Although specific laboratory tests for individual disease like antiglobulin test in autoimmune hemolytic anemia is available, early diagnosis of extravascular hemolysis is very difficult.

### SUMMARY OF THE INVENTION

This invention is related to the diagnostic method of hemolytic anemia, which allows early diagnosis of hemolytic anemia and discrimination of significant schistocytes from insignificant old schistocytes by utilizing the flow cytometric detection of damaged RBCs using anti-Hb in a hypotonic solution compared to saline solution, in the case of the presence of schistocytes in the peripheral blood, such as microangiopathic hemolytic anemia (MAHA).

This method does not require the washing or lysing steps, and get the result within 20 minutes easily. In detail, two microliter of peripheral blood isolated from a patient was stained with PE conjugated antihemoglobin (anti-Hb) antibody in 0.6% NaCl for 15 minutes at room temperature. Then, without wash, 3 ml of saline were added and analyzed by flow cytometry.

Therefore, compared with the counting schistocytes on the stained blood smears the time-consuming and hard to distinguish schistocytes from indented normal red cells (RBCs). This invention provides here a new quick and accurate diagnostic method of hemolytic anemia by flow cytometry using antihemoglobin (anti-Hb) antibody which detect only newly produced damaged RBCs and discriminate significant from insignificant old schistocytosis.

### DETAILED DESCRIPTION OF THE INVENTION

To solve the problems described above, this invention provides a new accurate and sensitive diagnostic method of hemolytic anemia.

This method does not require the washing or lysing steps, and get the result within 20 minutes easily. In detail, two microliter of peripheral blood isolated from a patient was stained with PE conjugated antihemoglobin (anti-Hb) antibody in 0.6% NaCl for 15 minutes at room temperature. Then, without wash, 3 ml of saline were added and analyzed by flow cytometry.

Therefore, compared with the counting schistocytes on the stained blood smears the time-consuming and hard to distinguish schistocytes from indented normal red cells (RBCs).this invention provides here a new quick and accurate diagnostic method of hemolytic anemia by flow cytometry using antihemoglobin (anti-Hb) antibody which detect only newly produced damaged RBCs and discriminate significant from insignificant old schistocytosis.

The following examples are intended to illustrate the invention.

This invention is related to the diagnostic method of hemolytic anemia including the steps of staining two microliter of peripheral blood isolated from a patient with PE conjugated antihemoglobin (anti-Hb) antibody in 0.6% NaCl for 15 minutes at room temperature, adding 3 ml of saline without wash, and analyzing the samples by flow cytometry.

To proceed the test, the following patients were studied; 70 anemia patients, 42 microangiopathic hemolytic anemia (MAHA), 8 malaria, 8 spherocytosis (3 hereditary spherocytosis, 3 autoimmune hemolytic anemia, 2 spherocytosis of unknown origin), 2 elliptocytosis, 6 postsplenectomy, 4 iron deficiency anemia (IDA), and 107 healthy adult platelet donors as normal control.

Forty of 42 patients with MAHA were acute or chronic leukemia patients and 19 of them received bone marrow transplantation (BMT).

Four of 6 postsplenectomy patients had splenectomy due to idiopathic thrombocytopenic purpura (ITP) 15 days to 5 years ago and remaining 2 patients had splenectomy due to huge splenomegaly by chronic myelogenous leukemia 2 months and 3 years ago.

In all 3 hereditary spherocytosis patients, samples from parents and siblings were also studied.

And the osmotic fragility tests of the patients showed hemolysis beginning from 0.52% to 0.62% sodium chloride©.

All malaria patients were infected with Plasmodium vivax.

EDTA anticoagulated blood samples for complete blood cell count (CBC) were used within 4 hours after collection. The samples were maintained at room temperature (18 to 20° C.) before analysis.

The following examples are intended to illustrate the invention utilizing the samples of patients described above.

Flow Cytometric Estimation Of Hemolysis

Damaged RBCs were not stained with antihemoglobin antibody (anti-Hb) in normal saline (FIG. 1). To find out the right concentration of sodium chloride in which hemoglobin in the damaged RBCs could be exposed to antihemoglobin antibody through damaged membranes, the peripheral bloods from 10 patients with MAHA and 10 normal persons were stained with anti-Hb antibody (Dako A/S, Denmark) in different concentrations of sodium chloride from 0.2% to 2.0% (FIG. 1). The right concentration which could discriminate MAHA from normal samples were 0.6% NaCl as the result.

Five normal and 5 MAHA samples were stained with antibody immediately, 1 day, 2 days, 3 days, 4 days, 6 days and 7 days after mix in 0.6% NaCl to check stability of the reagents. The antibody was labeled with phycoerythrin (PE) and diluted in phosphate buffered saline with 0.1% gelatin and penta-cholorophenol as preservative. The osmolarity of the antibody base solution was 316 mOsm/kg. Five microliter of antihemoglobin antibody was mixed with 50 μl of each NaCl solution.

The osmolarity of the antibody mixture in 0.6% NaCl was 213 mOsm/kg. The other samples were studied using antibody and 0.6% NaCl mixture. And 2 μl of peripheral blood was added to this antibody-NaCl mixture, and incubated for 15 minutes at room temperature.

Then just add 3 ml of saline without wash. Fluorescence was analyzed by flow cytometry (FACSCalibur. Beckton Dickinson) using CELLQuest software. Quality control of the flow cytometer was carried out twice a week using CaliBRITE™ beads (Becton Dickinson) and Autocomp software monthly.

The instruments setting of all channel were linear mode and the threshold level of forward scatter (FSC) was 52 and 20,000 cells were analyzed and stored. A large gate which includes all intact RBC and platelets was used (FIG. 1), because some of schistocytes were expected to smaller than intact RBCs.

All samples were stained with isotypic control antibody (Becton-Dickinson, San Jose, Calif.) to set markers also. Markers were set using isotypic control sera. To estimate precision of the method, 2 normal and 2 MAHA samples were analyzed ten times.

Microscopic Examination Of RBC Morphology

The blood smears from all samples were made and air dried. All slides were stained with Wright's stain and examined by two pathologists. The schistocytes were counted under the oil immersion fields (HPF) under the 1000×magnification, because the average number of RBCs in one HPF is 200 under the 1000×magnification. The number of schistocytes per HPFx ½ were used as a percentage of schistocytes in this study.

All the statistical data were analyzed using the independent samples T test. To evaluate correlation between the percentages of RBCs labeled with anti-Hb and the number of schistocytes counted by microscopic examination, the Pearson correlation coefficient and P value were calculated. The significance was evaluated using Wilcoxon Signed Ranks test using SPSS software.

Result Of Flow Cytometry In Serial NACL Concentration

More than 50% of RBCs in normal control and patients were stained with anti-Hb in 0.2% NaCl solution, and the proportion of stained RBC decreased upon increase of the concentration of NaCl up to 0.6% (FIG. 1). At the point of 0.6% NaCl, only MAHA samples showed stained RBCs more than 1% and all normal control samples showed less than 1% stained RBCs. At higher concentration of NaCl (0.7% to 2.0%), none of the samples of normal or MAHA patients were stained with anti-Hb.

Results Of Flow Cytometry In 0.6% NACL

The proportion of stained RBCs in each group was as Table 1.

TABLE 1
The proportion of red blood cells stained with anti-Hb antibody in
0.6% NaCl in each disease group.

| group | Number of cases | Mean ± SD (%) |
|---|---|---|
| normal control | 107 | 0.55 ± 0.23 |
| microangiopathic | 42 | 2.95 ± 2.95 |
| hemolytic anemia | | |
| malaria | 8 | 1.87 ± 0.72 |
| spherocytosis | 8 | 3.02 ± 1.12 |
| postsplenectomy | 8 | 0.78 ± 0.24 |
| Iron deficiency anemia | 4 | 0.59 ± 0.11 |

The reagents was stable up to 1 week and the proportion of stained RBCs was not decreased coefficient of variation was 15.0%. The proportion of stained RBCs in normal control was 0.55±0.23%, and only 7 out of 107 normal control revealed greater than 1% (1.01 to 1.12%) stained RBCs.

The proportion of stained RBCs in patients with MAHA was significantly higher than normal control (2.95±2.95%, P=0.000), and only one out of 42 MAHA samples revealed less than 1% stained RBCs. The number of schistocytes was 3.1±1.8% and was well correlated with the proportion of stained RBCs by anti-Hb (r=0.637, P=0.000). The proportion of stained RBCs in patients with malaria was significantly higher than normal control (1.87±0.72%, P=0.001), and the only case which showed less than 1%, stained RBCs revealed only one ring from Plasmodium on the slide.

The proportion of stained RBCs in patients with spherocytosis was significantly higher than normal control (3.02±1.12%, P=0.000), and all 8 samples revealed greater than 1% stained RBCs. There was no significant difference between hereditary spherocytosis and autoimmune hemolytic anemia. The results of flow cytometry were compared to the results of osmotic fragility tests.

Two hereditary spherocytosis cases began hemolysis at 0.52% and did not show hemolysis at 0.6% NaCl by osmotic fragility test, but 5.06% and 2.24% of RBCs were stained with anti-Hb in 0.6% NaCl. The other hereditary spherocytosis sample began hemolysis at 0.62% and 3.40% of RBCs were stained with anti-Hb in 0.6% NaCl, and the patient's father began hemolysis at 0.52% but 4.04% of RBCs were stained with anti-Hb in 0.6% NaCl.

Two cases showing marked elliptosis revealed 2.20 and 2.21% stained RBCs.

The proportion of stained RBCs in patients who had splenectomy before was similar to normal control (0.78±0.24%, P=0.069). Although many schistocytes(mean 4.42%) and burr cells were found in the peripheral blood (FIG. 3), the count of schistocytes was not correlated to the proportion of stained RBCs by anti-Hb (P=0.853).

All IDA cases showed less than 1% stained RBCs (0.59±0.11%).

How the fragmented red cells could circulate in the blood without loss of hemoglobin is unknown. Because the schistocytes should have loss of their cytoplasm, we tried to stain the schistocytes with anti-Hb antibody. But they were not stained in the isotonic solution. Therefore, we tried to expose hemoglobin through the temporary cover of the damaged cytoplasm by incubation in hypotonic solution and 0.6% NaCl solution was selected. In the more hypotonic solution, some of the normal RBCs were also stained with anti-Hb. It means normal RBCs could be damaged by incubation in hypotonic solution less than 0.6% NaCl. And incubation in hypertonic solution up to 2% NaCl did not stain the RBCs with anti-Hb in normal and MAHA samples. Shrink of RBCs could not expose hemoglobin through the damaged cytoplasm.

The proportion of stained RBCs in normal control was 0.55±0.23%, and that in patients with MAHA was significantly higher than normal control (2.95±2.95%, P=0.000), The number of schistocytes was well correlated with the proportion of stained RBCs by antiHb(r=0.637, P=0.000). This flow cytometric detection method does not need to wash or lysing steps and the results can be read within 20 minutes. Counting of the schistocytes in the blood smear is time consuming and frequently it is not easy to discriminate indented RBCs from true fragmented RBCs. Using this simple and rapid method, accurate estimation of schistocytes could be possible. Furthermore, as in the postsplenectomy cases, sometimes many schistocytes are found without any clinical evidence of intravascular hemolysis. And in such cases, it is impossible to determine the significance of schistocytosis by microscopic examination only. Using this flow cytometric method, the old schistocytes in the postsplenectomy patients were not detected. Because the damaged cells are eradicated from circulation by spleen, it is possible that the schistocytes in the postsplenectomy patients could survive and circulate in the blood for enough time to repair the damaged cytoplasm permanently.

This flow cytometric detection of schistocytes will be the only method to diagnose and monitor MAHA developed in postsplenectomy patients.

The proportion of stained RBCs in patients with malaria was significantly higher than normal control (1.87±0.72%, P=0.001). Malaria is a parasitic disease caused by Plasmodium. The RBCs are lysed during the course of the malaria. Using this method, we found that some of the damaged RBCs circulate in the blood. In the endemic area, routine screening of the febrile patients using this methods will be helpful to diagnose malaria.

The proportion of stained RBCs in patients with spherocytosis was significantly higher than normal control (3.02±1.12%, P=0.000), and all 8 samples revealed greater than 1% stained RBCs.

There was no significant difference between hereditary spherocytosis and autoimmune hemolytic anemia in the proportion of stained RBCs. The mechanism of spherocytic change has been studied and several defects in the structure of the cytoplasmic membrane have been reported. Such defects seem to large enough to pass the anti-Hb antibody. The laboratory tests to diagnose hereditary spherocytosis family, the patient's parents and 2 siblings showed normal osmotic fragilities, but the patient's father showed increased stained RBCs by anti-Hb. The results demonstrate this flow cytometric detection method of damaged cells are more sensitive than osmotic fragility test. Autoimmune diseases are a leading cause of death among young and middle-aged woman in the United States. Autoimmune hemolytic anemia occurs approximately 10% of patients with systemic lupus erythematosus, but it is possible that mild degrees of hemolysis are more common. It may be the sole presenting sign of the disease and may predate the appearance of other disease manifestation could be possible. The elliptocytosis is also very heterogeneous disases (William p543) and dysfunction or deficiency of membrane proteins have been reported (William p543 reference). All of 2 elliptocytosis cases showed increased stained RBCs by anti-Hb like spherocytosis. Although spherocytosis and elliptocytosis are found mainly in extravascular hemolysis, the result of this study demonstrate that these poikilocytes have large enough membrane defects to pass the anti-Hb antibody on a hypotonic solution.

All IDA cases and cases with tear-drop cells or target cells (data not shown) did not show increase of stained RBCs by anti-Hb. It means these poikilocytes do not have membrane defects sufficient to expose hemoglobin through the damaged cytoplasm. In conclusion, flow cytometric detection of damaged RBCs using anti-Hb in a hypotonic solution is a simple, sensitive and accurate method for detection of hemolysis. And it will be very helpful to make early diagnosis of hemolytic anemia, and discriminate significant schistocytosis from insignificant, old schistocytosis.

Industrial Effect

Therefore, this invention provides a new accurate and sensitive diagnostic method of hemolytic anemia by flow cytometry using antihemoglobin (anti-Hb) antibody in hypotonic saline solution, which detect only newly produced damaged RBCs, discriminate significant from insignificant old schistocytosis, and detect the progress of hemolysis in the patients of hereditary disease, pregnancy toxemia, cancer, bone marrow transplantation, septicemia, and burn.

What is claimed:

1. Diagnostic Method of Hemolytic Anemia, which detect the number of damaged RBCs and discriminate newly produced significant shistocytes from insignificant old schistocytosis, including the steps of:
staining two microliter of peripheral blood isolated from a patient with PE conjugated antihemoglobin (anti-Hb) antibody in 0.6% NaCl for 15 minutes at room temperature,
adding 3 ml of saline without wash, and
analyzing the samples by flow cytometry.
